(19) United States
(12) Patent Application Publication
(10) Pub. No.: US 2019/0249217 A1
(43) Pub. Date: Aug. 15, 2019

(54) SYNTHETIC SWEAT COMPOSITION, SWEAT ODOR KIT, AND METHOD OF USE

(71) Applicant: Microban Products Company, Huntersville, NC (US)

(72) Inventors: Katherine Hawley, Huntersville, NC (US); Gina Parise Sloan, Statesville, NC (US); Glenner Marie Richards, Davidson, NC (US); Brian Patrick Aylward, Concord, NC (US); Karen Terry Welch, Kannapolis, NC (US); Tian Lan, Huntersville, NC (US); Siqi Li, Charlotte, NC (US)

(21) Appl. No.: 16/271,335

(22) Filed: Feb. 8, 2019

Related U.S. Application Data
(60) Provisional application No. 62/630,007, filed on Feb. 13, 2018.

(51) Int. Cl. C12Q 1/18

(52) U.S. Cl. CPC C12Q 1/18

(57) ABSTRACT

A synthetic sweat composition, sweat odor kit, and method of use are provided. The synthetic sweat composition comprises an odor-precursor of eccrine sweat and an odor pre-cursor of apocrine sweat. The method comprises combining a synthetic sweat composition with a mixture of body odor causing microorganisms or bacteria, applying the combination to a textile, incubating the textile, and establishing an odor measurement to rate the odor of each textile.

### CROSS-REFERENCE TO RELATED APPLICATIONS

This application claims priority from U.S. provisional patent application No. 62/630,007, filed on Feb. 13, 2018, in the United States Patent and Trademark Office. The disclosure of which is incorporated herein by reference in its entirety.

### FIELD OF THE INVENTION

The present invention relates to a synthetic sweat composition, sweat odor kit, and method of use.

### BACKGROUND OF THE INVENTION

Artificial or synthetic sweat compositions are used for biological research in human health, for industrial research in odor mitigation for textiles, and for industrial research for personal care products. Many such compositions are used to ascertain colorfastness to perspiration, such as the AATCC 15 artificial sweat test method (AATCC Test Method 15-2007, “Colorfastness to Perspiration”). While these artificial sweat compositions do contain many of the components of human eccrine sweat, they neglect components of apocrine sweat and normal skin microflora that are responsible for human malodor. Some recent formulations of artificial sweat have attempted to correct this omission. These compositions are mostly intended for the cosmetic and deodorant industries. However, these compositions are extremely complex, and some contain immediate human by-products such as fats obtained from liposuction surgeries and axillary swabs (Harvey, C. J. et al. (2010) Formulation and stability of a novel artificial human sweat under conditions of storage and use. Toxicol. In Vitro 24, 1790-1796; Callewaert, C. et al. (2014) Artificial sweat composition to grow and sustain a mixed human axillary microbiome. Journal of Microbiological Methods 103, 6-8).

While these compositions are most closely similar to actual human sweat, they are too complex and potentially dangerous since human body wastes are involved.

There are no tests that specifically evaluate odor produced by microorganisms in fabrics that simulate “in use” conditions. Wear trials can be used to evaluate odor build up on textiles, but they are costly, time consuming and suffer from variation due to individual odor profiles of the participants.

Thus, there is a need for a rapid, simple but yet more comprehensive artificial sweat that would allow for survival of bacteria associated with human malodor and that would encourage their normal metabolic processes that produce characteristic human malodor.

### SUMMARY OF THE INVENTION

The present invention relates to a synthetic sweat composition, sweat odor kit, and method of use.

In an embodiment of the invention, a synthetic sweat composition generally comprises an odor-precursor of eccrine sweat, and an odor pre-cursor of apocrine sweat.

In an embodiment of the invention, a synthetic sweat composition generally comprises a salt, a weak acid, a buffer system, an amino acid, a protein rich medium, a rich source of nutrient, urea, and a steroidal sweat compound.

In an embodiment of the invention, a sweat odor kit is provided. The sweat odor kit of the present invention comprises a synthetic sweat composition having nutrients and metabolites typical of apocrine sweat, and a mixture (also referred to as a cocktail) of bacteria or microorganisms associated with human skin microflora that are responsible for body odor. The kit provides for odor generation by the associated bacteria in order to evaluate odor abatement, for example, on textiles treated with antimicrobials or odor capture technology.

In an embodiment of the invention, the sweat odor kit comprises: (a) a synthetic sweat composition comprised of: a salt, a weak acid, a buffer system, an amino acid, a protein rich medium, a rich source of nutrient, urea, and a steroidal sweat compound; and (b) a mixture of odor causing microorganisms.

In an embodiment of the invention, the sweat odor kit comprises: (a) a sweat odor composition comprised of NaCl, lactic acid, Na₂HPO₄, 1-histidine monohydrochloride (C₆H₉N₃O₂.HCl.H₂O), a protein rich medium, a rich source of nutrient, urea, and a steroidal sweat compound; and (b) a mixture of odor causing microorganisms or bacteria selected from the group consisting of Corynebacterium jeikeium, Corynebacterium striatum, Corynebacterium xerosis, Staphylococcus epidermidis, BHIB media, Micrococcus luteus, Propionibacterium, Staphylococcus saprophyticus, Proteus vulgaris, Staphylococcus aureus, and a combination thereof.

In an embodiment of the invention, a method of odor evaluation using the synthetic sweat composition and/or synthetic sweat kit is provided. The method generally comprises combining a synthetic sweat composition with a mixture of body odor causing bacteria or microorganisms, applying the combination to a textile, incubating the textile, and establishing an odor measurement (including, but not limited to, an odor panel, GC-head space analysis or an alternative analytical technique) to rate the odor of each textile.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is a graphical representation of a polyester fabric synthetic sweat odor intensity panel rating.

### DESCRIPTION OF THE PREFERRED EMBODIMENTS

The following description of the embodiments of the present invention is merely exemplary in nature and is in no way intended to limit the invention, its application, or uses. The present invention has broad potential application and utility, which is contemplated to be adaptable across a wide range of industries. The following description is provided herein solely by way of example for purposes of providing an enabling disclosure of the invention but does not limit the scope or substance of the invention.

Synthetic Sweat Composition

In an embodiment of the invention, a synthetic sweat composition comprises an odor-precursor of eccrine sweat, and an odor pre-cursor of apocrine sweat.

In an embodiment of the invention, a synthetic sweat odor composition generally comprises a salt (including, but not limited to, NaCl, CaCl₂, and MgCl₂), a weak acid relevant to sweat secretions (including, but not limited to, lactic acid, and acetic acid), a buffer system (including, but not limited to, phosphate or Tris buffers), an amino acid (including, but not limited to, histidine, cysteine, and phenylalanine), a rich nutrient source (including, but not limited to, Brain Heart Infusion Broth (BHIB), Tryptic Soy Broth (TSB)), a protein rich medium (including, but not limited to, Bovine Serum Albumin (BSA), Fetal Bovine Serum (FBS)), urea, and a steroidal sweat compound.

In an embodiment of the invention, the synthetic sweat odor composition comprises: 0.5% to 10% (weight/weight) of a salt, 0.05% to 5% (w/w) of a weak acid relevant to sweat secretions, 0.05% to 5% of a buffer system, 0.01% to 2% of an individual amino acid, 0.001% to 0.1% protein rich medium such as Bovine Serum Albumin or Fetal Bovine Serum, 0.05% to 2% urea, 0.0005% to 0.01% of a steroidal sweat compound such as 5,16-androstadien-3β-ol, and 1% to 5% of a rich source of nutrient (Brain Heart Infusion Broth, Tryptic Soy Broth, etc.).

In an embodiment of the invention, the synthetic sweat composition comprises: 0.5% to 10% (w/w) NaCl, 0.05% to 5% (w/w) lactic acid, 0.05% to 5% Na₂PO₄, 0.01% to 2% (w/w) L-histidine, 0.001% to 0.1% Bovine Serum Albumin, 0.05% to 2% urea; 0.0005% to 0.01% 5,16-androstadien-3β-ol, and 1% to 5% Brain Heart Infusion Broth.

The synthetic sweat odor composition of the present invention preferably has a pH in a range of 4 to 5.

Sweat Odor Kit

In an embodiment of the invention, a sweat odor kit is provided. The sweat odor kit of the present invention comprises: a synthetic sweat composition having nutrients and metabolites typical of apocrine sweat, and a mixture (also referred to as a cocktail) of bacteria associated with human skin microflora that are responsible for body odor.

In accordance with an embodiment of the invention, the cocktail of microorganisms is typical to those found on human skin. A non-limiting example of such odor causing microorganisms is selected from the group consisting of Corynebacterium jeikeium, Corynebacterium striatum, Corynebacterium xerosis, Staphylococcus epidermidis, BHIB media, Micrococcus luteus, Propionibacterium, Staphylococcus saprophyticus, Proteus vulgaris, Staphylococcus aureus, and a combination thereof. The microorganism cocktail can be present within a range of 10⁴ to 10⁷ CFU/ mL.

In accordance with an embodiment of the invention, the sweat odor kit comprises: (a) a sweat odor composition comprised of NaCl, lactic acid, Na₂HPO_{4, 1}-histidine monohydrochloride (C₆H₉N₃O₂.HCl.H₂O), a protein rich medium, a rich source of nutrient, urea, and a steroidal sweat compound; and (b) a mixture of odor causing microorganisms or bacteria selected from the group consisting of Corynebacterium jeikeium, Corynebacterium striatum, Corynebacterium xerosis, Staphylococcus epidermidis, BHIB media, Micrococcus luteus, Propionibacterium, Staphylococcus saprophyticus, Proteus vulgaris, Staphylococcus aureus, and a combination thereof.

The kit provides for odor generation by the associated microorganisms in order to evaluate odor abatement, for example, on textiles treated with antimicrobials or odor capture technology.

Method for Odor Evaluation

In an embodiment of the invention, a method for odor evaluation is provided to test the ability of artificial sweat to allow survival and odor generation though normal metabolic processes of the bacteria. The method generally comprises introducing the sweat composition with microorganisms onto test fabric to evaluate the odor present after an incubation period. Utilizing the synthetic sweat odor composition of the present invention and microorganism cocktail, it is possible to use the method of the present invention to identify textile treatments that could provide wearers with less odor in their fabrics after use.

In an embodiment of the invention, a method for odor evaluation is provided. The method generally comprises combining a synthetic sweat composition with a mixture of body odor causing bacteria or microorganisms, applying the combination to a textile, incubating the textile, and establishing an odor measurement (including, but not limited to, an odor panel, GC-head space analysis or an alternative analytical technique) to rate the odor of each textile.

The method and the synthetic sweat odor kit of the present invention can be used to evaluate textile treatments quickly and inexpensively in a laboratory-controlled setting to show odor reduction caused by microorganisms found on human skin.

Among the advantages of the method and kit of the present invention are that they provide a rapid, consistent and inexpensive alternative to wear studies to assess anti-odor treatments on textiles. The synthetic sweat composition mimics many components of human sweat including the microorganisms found on skin, particularly the axillary region. Utilizing the kit of the present invention provides a method of evaluating and scoring the odor evolving from the fabrics exposed to the synthetic sweat odor during the incubation period. By utilizing the method of the present invention instead of a wear trial, time and money can be saved. Due to the unique body chemistries and odor profiles of humans, large groups of wearers would be required to achieve the consistent results a laboratory evaluation can provide.

### EXAMPLE 1

The synthetic sweat odor composition of the present invention was prepared as follows.

A pre-mix of the following compounds was made (“AATCC 15 sweat”) was made prior to testing and autoclaved (121° C./15 psi/20 minutes). The AATCC 15 sweat was removed in aliquots to make the synthetic sweat odor composition of the present invention used in the evaluation. The aliquot of AATCC 15 sweat was added to a sterile tube. To each aliquot was added: 1 mg/ml Bovine Serum Albumin (from a 100 mg/ml stock; 0.1% urea (from a 10% stock); 100 ug/ml 5,16-androstadien-3β-ol in DMSO (from a 25 mg/ml stock); Brain Heart Infusion Broth containing sweat microorganism cocktail (see below in Microorganism Preparation) to a 2% concentration in the final sweat odor cocktail. This sweat cocktail simulated nutrients found on the human body such as dead skin cells.

Microorganism cocktail preparation: Test organisms were prepared by inoculating separate plates containing Tryptic Soy Agar with one inoculation loop full of the appropriate stock culture and incubated for at least 24 hours at 37°±2° C. until colonies were visible. Plates can be stored at 4°±2° C. for 1 month.

Following incubation, the inoculum for the study was prepared as follows. An individual CFU was taken on an inoculation loop of each of the sweat organisms (Corynebacterium jeikeium, Corynebacterium striatum (2 strains), Corynebacterium xerosis, and Staphylococcus epidermidis) and added to a tube with 9 ml of BHIB. These organisms were added to the artificial sweat composition to achieve a 2% concentration of nutrient. The total number of organisms in the final synthetic sweat composition was checked to be in the 10̂5 to 10̂6 cfu/ml range.

Odor Evaluation Procedure:

For purposes of odor evaluation, synthetic sweat was pipetted directly onto fabrics with odor treatments. The fabric was secured in a closed container and samples were incubated at 36° C. for 48-72 hours, or until a sweat-like odor was discernable in the untreated or control fabric sample.

After the challenge time, usually 48-72 hours, an odor panel was established to rate the odor of each fabric in the vials on a scale of 0-10, with 0 being no odor and 10 being a strong, unpleasant odor. The odor panel included at least 5 individuals that have not been associated with the testing.

After the odor panel was evaluated the fabrics for odor.

The sweat kit of the present invention has been used to evaluate anti-odor treatments on a variety of fabrics and treatments. It has been shown that bacterial populations correspond to odor panel scores in positive relationship.

### EXAMPLE 2

1. Sweat Odor Composition:

The sweat odor utilized a base of eccrine type sweat (for example the acid perspiration in AATCC 15, see formulation below). This eccrine sweat odor formulation was made prior to testing and autoclaved (121° C./15 psi/20 minutes).

Eccrine base sweat formulation per 1 L deionized H₂O:

10 g NaCl

1 g Lactic Acid

1 g Na₂HPO4

0.25 g 1-histidine monohydrochloride (C6H9N3O2.HCl.H2O)

pH should be 4.3±0.2

Eccrine base sweat was removed in aliquots to make the total synthetic sweat odor cocktail used in the evaluation. The aliquot of Eccrine base sweat was added to a sterile tube. To each aliquot was added:
1 mg/ml (0.01%) Bovine Serum Albumin (from a 100 mg/ml stock)
0.1% urea (from a 10% stock)
100 ug/ml (0.001%) 5,16-androstadien-3β-ol in DMSO (from a 25 mg/ml stock)
Brain Heart Infusion Broth containing sweat microorganism cocktail (see below in Microorganism Preparation) to a 2% concentration in the final sweat odor cocktail. This simulates nutrients found on the human body such as dead skin cells.

2. Microorganism Cocktail Preparation:

A test-organism cocktail was prepared by inoculating separate plates containing Tryptic Soy Agar with one inoculation loop full of the appropriate stock culture and incubated for at least 24 hours at 37°±2° C. until colonies were visible. Plates were stored at 4°±2° C. for 1 month. Following incubation, the inoculum was prepared for the study as follows: an individual CFU was taken on an inoculation loop of each of the sweat organisms (Corynebacterium jeikeium, Corynebacterium xerosis, Corynebacterium striatum (2 strains), and Staphylococcus epidermidis) and added to a tube with 9 ml of BHIB. These organisms were added to the artificial sweat mixture (detailed above) to achieve a 2% concentration of nutrient. The total number of organisms in the final sweat odor cocktail was in the 10̂5 to 10̂6 cfu/ml range.

3. Odor Evaluation Procedure:

a. Synthetic sweat was pipetted directly onto fabrics with odor treatments. The fabric was secured in a closed container and samples were incubated at 36° C. for 48-72 hours, or until a sweat-like odor was discernable in the untreated or control fabric sample.

b. After the challenge time, usually 48-72 hours, an odor panel was established to rate the odor of each fabric in the vials on a scale of 0-10, with 0 being no odor and 10 being a strong, unpleasant odor. The odor panel included at least 5 individuals that have not been associated with the testing.

c. After the odor panel evaluated the fabrics for odor, if organism enumeration was desired that was be carried out in the preferred manner.

### EXAMPLE 3

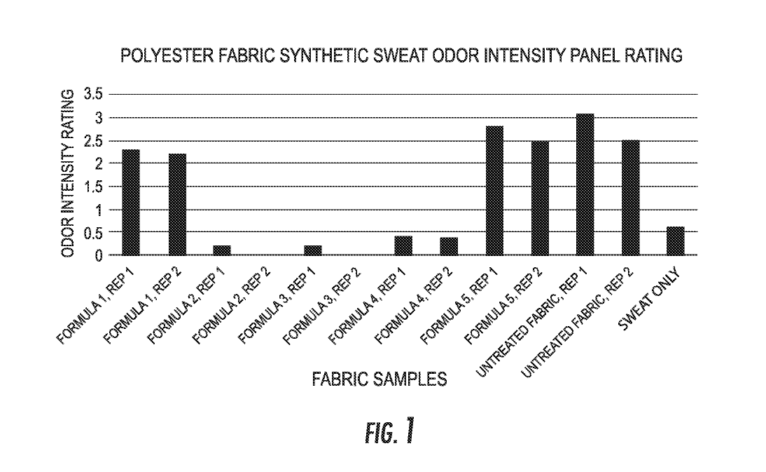

To examine if the sweat odor formulation works, a polyester fabric was treated with a variety of odor control formulations at various concentrations and the fabrics were tested with the sweat formula and the procedure as referenced in Example 2. The odor intensity rating is shown in FIG. 1. The odor intensity was rated with a 10-point scale from 0 to 10 with 0 being no odor and 10 being extremely strong odor. FIG. 1 clearly shows that the odor intensities rated from fabric are different among fabrics dependent upon the odor control formulation. The odor intensities were also found to be consistent among most replicates indicating that the test was a reliable and repeatable test for fabric odor control performance assessment.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A synthetic sweat composition comprising:
an odor-precursor of eccrine sweat, and
an odor pre-cursor of apocrine sweat.

2. A synthetic sweat composition comprising:
a salt,
a weak acid,
a buffer system,
an amino acid,
a protein rich medium,
a rich source of nutrient,
urea, and
a steroidal sweat compound.

3. The synthetic sweat composition according to claim 2, wherein the salt is selected from the group consisting of NaCl, CaCl₂, MgCl, and a combination thereof.

4. The synthetic sweat composition according to claim 2, wherein the weak acid is selected from the group consisting of lactic acid, acetic acid, and a combination thereof.

5. The synthetic sweat composition according to claim 2, wherein the buffer system is selected from the group consisting of phosphate, Tris buffers, and a combination thereof.

6. The synthetic sweat composition according to claim 2, wherein the amino acid is selected from the group consisting of histidine, cysteine, phenylalanine, and a combination thereof.

7. The synthetic sweat composition according to claim 2, wherein the protein rich medium is selected from the group consisting of Bovine Serum Albumin (BSA), Fetal Bovine Serum, and a combination thereof.

8. The synthetic sweat composition according to claim 2, wherein the rich source of nutrient is selected from the group consisting of The synthetic sweat composition according to claim 2, wherein the Brain Heart Infusion Broth (BHIB), Tryptic Soy Broth (TSB), and a combination thereof.

9. The synthetic sweat composition according to claim 2, wherein the synthetic sweat odor composition comprises 0.5% to 10% of a salt.

10. The synthetic sweat composition according to claim 2, wherein the synthetic sweat odor composition comprises 0.05% to 5% of a weak acid relevant to sweat secretions.

11. The synthetic sweat composition according to claim 2, wherein the synthetic sweat odor composition comprises 0.05% to 5% of a buffer system.

12. The synthetic sweat composition according to claim 2, wherein the synthetic sweat odor composition comprises 0.01% to 2% of an individual amino acid.

13. The synthetic sweat composition according to claim 2, wherein the synthetic sweat odor composition comprises 0.001% to 0.1% protein rich medium

14. The synthetic sweat composition according to claim 2, wherein the synthetic sweat odor composition comprises 0.05% to 2% urea.

15. The synthetic sweat composition according to claim 2, wherein the synthetic sweat odor composition comprises 0.0005% to 0.01% of a steroidal sweat compound.

16. The synthetic sweat composition according to claim 2, wherein the synthetic sweat odor composition comprises 1% to 5% of a rich source of nutrient.

17. A sweat odor kit comprising:
a synthetic sweat composition comprised of:
a salt,
a weak acid,
a buffer system,
an amino acid,
a protein rich medium,
a rich source of nutrient,
urea, and
a steroidal sweat compound;
and
a mixture of odor causing microorganisms.

18. A sweat odor kit comprising:
a sweat odor composition comprised of:
NaCl,
lactic acid,
Na₂HPO₄,
1-histidine monohydrochloride (C₆H₉N₃O₂.HCl.H₂O),
a protein rich medium,
a rich source of nutrient,
urea, and
a steroidal sweat compound;
and
a mixture of odor causing microorganisms or bacteria selected from the group consisting of Corynebacterium jeikeium, Corynebacterium striatum, Corynebacterium xerosis, Staphylococcus epidermidis, BHIB media, Micrococcus luteus, Propionibacterium, Staphylococcus saprophyticus, Proteus vulgaris, Staphylococcus aureus, and a combination thereof.

19. A method comprising:
combining a synthetic sweat composition with a mixture of body odor causing microorganisms or bacteria,
applying the combination to a textile,
incubating the textile, and
establishing an odor measurement to rate the odor of each textile.

20. The method according to claim 19, wherein the synthetic sweat composition comprises an odor-precursor of eccrine sweat and an odor pre-cursor of apocrine sweat.

21. The method according to claim 19, wherein the synthetic sweat composition comprises a salt, a weak acid, a buffer system, an amino acid, a protein rich medium, a rich source of nutrient, urea, and a steroidal sweat compound.

22. The method according to claim 19, wherein the synthetic sweat composition comprises NaCl, lactic acid, Na₂HPO₄, 1-histidine monohydrochloride (C₆H₉N₃O₂.HCl.H₂O), a protein rich medium, a rich source of nutrient, urea, and a steroidal sweat compound.

23. The method according to claim 19, wherein the mixture of odor causing microorganisms or bacteria selected from the group consisting of Corynebacterium jeikeium, Corynebacterium striatum, Corynebacterium xerosis, Staphylococcus epidermidis, BHIB media, Micrococcus luteus, Propionibacterium, Staphylococcus saprophyticus, Proteus vulgaris, Staphylococcus aureus, and a combination thereof.
